(19) United States
(12) Patent Application Publication
(10) Pub. No.: US 2010/0083406 A1
(43) Pub. Date: Apr. 1, 2010

(54) Black Garabedian

(76) Inventor: Silvestre Arias, Fresno, CA (US)

Correspondence Address: MARK D MILLER;KIMBLE, MACMICHAEL & UPTON, 5260 NORTH PALM AVENUE, SUITE 221, FRESNO, CA, 93704, US

(21) Appl. No.: 12/242,289

(22) Filed: Sep. 30, 2008

(51) Int. Cl. A01H 5/00

(52) U.S. Cl. 800/298

(57) ABSTRACT

A new and distinct variety of plum tree that is characterized by producing a semi freestone fruit with good dark red coloration and is ripe for commercial harvesting and shipment June 3-June 14 in the San Joaquin Valley of Central Calif. The new variety is closely similar to ‘Santa Rosa’ plum (non patented) from which it is hybridized but it is distinguishable in that it ripens seven to ten days before ‘Santa Rosa’ variety. The fruit of this new variety is medium size with good flavor and good dark red color which makes it greatly acceptable for the consumer.

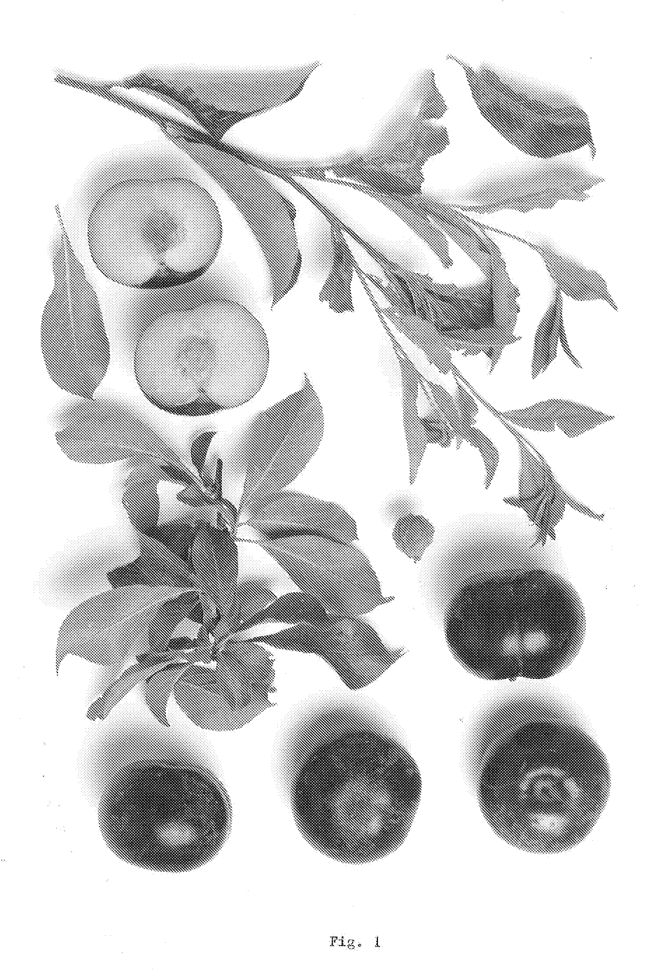

### BACKGROUND OF THE NEW VARIETY

The present invention refers to a new variety of plum which will hereinafter be denominated as the ‘Black Garabedian’ plum tree. The ‘Black Garabedian’ is a medium size dark red fruit with yellow flesh that is mature for harvesting and shipping in the June 3 to June 14 period in a normal year in the San Joaquin Valley of Central Calif.

In the development of new commercial varieties specific characteristics provide a premium on those fruit varieties that mature early or late in the growing season. However, in particular early varieties lack size, flavor and coloration on blush. Therefore, in order for a fruit to be a success, it must be of good size, have flavor, good color, and also have good holding/storage and shipping characteristics. In addition, the date of maturing must be different from other similar fruit. This new invention meets all of the aforementioned criteria and therefore would be of commercial appeal to the consumer.

Origin and Asexual Reproduction of the New Variety

The present invention (variety) was discovered by the inventor, Silvestre Arias, on a varietal test block near the city of Fresno, Calif. The invention was discovered as a hybrid of ‘Santa Rosa’ (non patented) and ‘El Dorado’ (non patented) plum trees. The new variety was asexually reproduced in the dormant season of 2005 by bud grafting 60 acres (1200 trees) on Nemared rootstock about seven to eight miles from the original planting. The inventor carefully compared the asexually reproduced trees with the parent hybrid including the fruit and found that they are identical in all respects.

### SUMMARY OF THE NEW VARIETY

‘Black Garabedian’ plum tree is characterized by producing a medium semi-free stone fruit which has a very good blush coloration and is ripe for commercial eating and shipping around June 3 to June 15 in a normal year in the San Joaquin Valley of Central Calif. as a medium fruit with dark red blush and yellow flesh. The new variety is most similar to ‘Santa Rosa’ plum tree (non patented) from which it is a prodigy but distinguishable in that it ripens seven to ten (7-10) days before the ‘Santa Rosa’ variety. The fruit of this new variety is medium size with good flavor and good dark red color which makes it greatly acceptable to the consumer.

### BRIEF DESCRIPTION OF THE DRAWINGS

The accompanying colored photograph shows the following: foliage including stem and new growth at the upper right; below this foliage is a fruit cut in half displaying the pit well in the upper half and the pit in the well in the lower half; below the cut fruit is additional terminal foliage; between the two sets of foliage is a pit; to the right of the pit is a side view of a ripened fruit; and along the bottom, from left to right, is another side view, a bottom view and a top view of a ripened fruit.

### DETAILED DESCRIPTION

Referring more specifically to the horticultural details of the new and distinct variety of plum trees, the following descriptions have been observed under the ecological conditions prevailing at the origin orchard near Fresno, Calif. in the San Joaquin Valley of Central Calif. All major color designations are by reference to the Dictionary of Color by Maerz and Paul, First Edition, published in 1930. Common colors are also employed.

### Tree

Size—medium, for third leaf tree

Height: 10-11 feet (304.8-335.3 cm)

Width: 8-9 feet (243.8-274.3 cm)

Vigor: good

Shape: pyramid—upright

Productivity: very good for third leaf tree

Regularity of bearing: regular

Chilling requirements: normal for plum trees in the Central San Joaquin Valley of Calif.

### Trunk

Size: medium—15.0 inches (38.1 cm) circumference, 14.0 inches (35.6 cm) above ground
Surface Texture: rough
Color Code: Vineyard Operto+(55-L-12)
Lenticels:
   Number: many
   Size: small 0.08-0.16 inch (2.0-4.0 mm)

### Branches

Size: medium—6.0-8.0 inches (15.2-20.3 cm) in circumference at approximately 3 years
Angle of branches: 52° from vertical
Color:
   One year or older: Wineberry (55-H-4)
   Less than one year old: Fern (21-I-7)

### Leaves

Size: medium
   Length: 3.15-3.94 inches (8.0-10.0 cm)
   Width: 1.38-1.77 inches (3.5-4.5 cm)
Shape: ovate
Margin: double serration
Apex: broadly acute
Base: broad
Surface Texture:
   Upwardly disposed surface: smooth
   Downwardly disposed surface: slightly rough
Glands: 2 small
   Color: green
Stipules: 1-3
   Color: Maroon (7-L-7)
   Upwardly disposed surface: Chrome Gr. Deep (24-H-12)
   Downwardly disposed surface: Art Gr. (22-L-7)
   Young terminal leaves display with: Rhododendron (54-J-6)
Petiole:
   Length: 0.43-0.59 inch (1.1-1.5 cm)
   Diameter: 0.08 inch (0.2 cm)
   Color: Fern (21-I-7)
Midvein: 0.04 inch (1.5 mm)
   Color: Rainette Gr. (21-K-4)

### Flowers

Flower Buds:
   Shape: conic—0.06-0.20 inch (1.5-5.0 mm)
   Size: Length: 0.16-0.20 inch (4.0-5.0 mm)
      Width: 0.12-0.20 inch (3.0-5.0 mm)
Calyx—5 sepals
   Color: (19-J-8), sl. darker than Calliste Gr. (19-L-6)
Date of First Bloom:
   Feb. 10, 2008—10-15%
   Feb. 28, 2008—100%
   Flowers generally showy
      Size: small, in clusters of 3-4
      Diameter when fully opened: average 0.71 inch (18.0 mm)
      Quantity: abundant
Petals: 5
   Size—small
      Length: 0.31-0.39 inch (8.0-10.0 mm)
      Width: 0.28-0.35 inch (7.0-9.0 mm)
   Form: broadly ovate
   Color: white
   Claws: small—0.04 inch (1.0 mm) truncate
   Petal Margins: undulate
   Pedicel: average 0.39-0.51 inch (10.0-13.0 mm)
   Sepals: Color—(19-J-8), darker than Calliste Gr. (19-L-6)
   Stamens: 16-20
   Filament; Color—white
   Anthers: small
      Color: Nugget Bronze Y (11-L-8)
   Pistil: longer than stamens
      Color: Sky Cr. (18-I-5)
   Fertility:
      Pollinators: Showtime—Santa Rosa

### Fruit

Date of Maturity: June 3-June 14
Size: medium
   Weight: 4 fruit—1.5 pounds (42.5 grams)
   Diameter of axial plane—1.97-2.05 inch (50-52 mm)
   Transverse in suture plane—2.44-2.64 inch (62-67 mm)
   Transverse at right angle to suture plane—2.64-2.68 inch (67-68 mm)
Form: nearly uniform
Stem cavity:

Length: 0.71-0.79 inch (18-20 mm)
Width: 0.71 inch (18 mm)
Depth: 0.31 inch (8 mm)
Stem:
   Length: 0.47-0.51 inch (12-13 mm)
   Width: 0.08 inch (2 mm)
   Apex: rounded
   Pistil Point: unnoticeable
   Skin: thickness normal for plum
   Texture: firm
Color:
   Blush: Burgundy (56-E-8)
   Ground: Dark Cardinal Rose lustre (6-L-8)
   Ground: Lime Y (11-L-5)
   Flesh:
      Near skin: Chartreuse (11-L-1)
      Remainder: Pyrethrum Y. (11-L-2)
   Surface of cavity: Burnished gold (12-L-7)
   Pit Well: Burnished gold (12-L-7)
Pit Well:
   Length: 0.91 inch (23 mm)
   Width: 0.71 inch (18 mm)
   Depth: 0.31-0.35 inch (8-9 mm)
Use: fresh market
Keeping quality: excellent
Harvest date: June 3-June 14

### Stone

Semi Freestone
Fibers: few
Size:
   Length: 0.91 inch (23 mm)
   Width: 0.79 inch (20 mm)
   Thickness: 0.47 inch (12 mm)
Form: ovate
Apex shape: somewhat rounded
Color (dry)—Chrome Op (10-K-12)
Base: somewhat flattened
Sides:
   Ventral: slightly winged
   Dorsal: ridged from apex to base
Tendency to split: none observed

The above description of this new variety of plum tree is based on the growing conditions prevalent in the Fresno area of the San Joaquin Valley of Central Calif. Variations of the usual magnitude, such as maturity date and production may be due to cultural practices including irrigation practices, fertilization, fruit planning and primary climate changes.

What is claimed is:

1. A new distinct plum tree as described and illustrated that is similar to the ‘Santa Rosa’ plum (not patented) which is one of the parents, but is distinguished in that the fruit is somewhat larger, has more dark red skin coloration with excellent flavor and stores well, maturing seven to ten days before ‘Santa Rosa’; and is also similar to ‘Black Amber’ (non patented) but has yellow flesh compared to ‘Black Amber’ and matures four to seven days before ‘Black Amber’.
